(19) United States
(12) Plant Patent Application Publication
(10) Pub. No.: US 2002/0199226 P1
(43) Pub. Date: Dec. 26, 2002

(54) Tiarella plant named 'Springwood'

(76) Inventors: Stephen K. Lighty, Cochranville, PA (US); Angela J. Treadwell, Baltimore, MD (US)

Correspondence Address: BURNS, DOANE, SWECKER & MATHIS, L.L.P., P.O. Box 1404, Alexandria, VA, 22313-1404, US

(21) Appl. No.: 09/875,926

(22) Filed: Jun. 8, 2001

(51) Int. Cl. A01H 5/00

(52) U.S. Cl. PLT/263

(57) ABSTRACT

A new variety of Tiarella cordifolia var. collina plant is provided that is well suited for growing as a distinctive ground cover. The new variety exhibits a compact mounding clump growth habit with the substantial absence of runners. Attractive white flowers are formed on unbranched flower stalks. The lobed leaves are ovate in configuration and possess a matte finish. During the summer the leaves are green and bear somewhat maroon centers. During the fall, the leaves assume a red coloration of variable intensity. The available choices of ornamental ground covers are expanded.

### SUMMARY OF THE INVENTION

The new Tiarella cordifolia var. collina variety was discovered during 1996 as a found seedling in the garden of Richard W. Lighty at Kennett Square, Pennsylvania, U.S.A. The exact parentage of the new variety is unknown. A variable population of the species was growing nearby that included Tiarella cordifolia, Tiarella cordifolia var. collina, and Tiarella cordifolia Wherryi. Some or all of these plants are likely included in the parentage of the new variety of the present invention.

The new variety has been carefully preserved and studied since the time of its discovery. Had such new variety not been discovered and preserved, it would have been lost to mankind.

It was found that the new Tiarella cordifolia, var. collina variety of the present invention exhibits the following combination of characteristics:

(a) exhibits a compact mounding clump growth habit with the substantial absence of runners,

(b) forms attractive white flowers on unbranched flower stalks,

(c) forms lobed ovate green leaves having a matte finish during the summer that bear somewhat maroon centers and turn red of variable intensity during the fall, and

(d) is particularly well suited for growing as a distinctive ornamental ground cover.

The new variety of the present invention can be readily distinguished from other previously known varieties of the species in view of the distinctive combination of characteristics discussed herein. The red fall color is considered to be particularly noteworthy.

The new variety well meets the needs of the horticultural industry and expands the choices of ornamental ground covers. It performs well wherever a ground cover is desired, and is particularly well suited for use as a border planting, use in shaded areas, and inclusion in a rock garden.

The division of clumps has been used to asexually propagate the new variety at West Grove, Pa., U.S.A. It has been found that the distinctive combination of characteristics of the new variety is firmly fixed and is reliably transmitted to succeeding generations. During observations to date, the new variety has been found to be readily amenable to such propagation.

The new variety has been named ‘Springwood’.

### BRIEF DESCRIPTION OF THE PHOTOGRAPHS

The accompanying photographs show specimens of the new variety in color as nearly true as is reasonably possible to make the same in color illustrations of this character. The plants were approximately two years of age and were being grown outdoors on their own roots at West Grove, Pa., U.S.A.

FIG. 1 illustrates a typical overall plant of the new variety while flowering during the summer. The commonly displayed maroon centers of the foliage are not readily depicted in the photograph.

FIG. 2 illustrates the typical red coloration of the leaves having variable intensity that is displayed during the fall.

FIG. 3 illustrates a closer view of flowers and foliage during the summer. The commonly displayed maroon centers of the foliage are not readily depicted in the photograph.

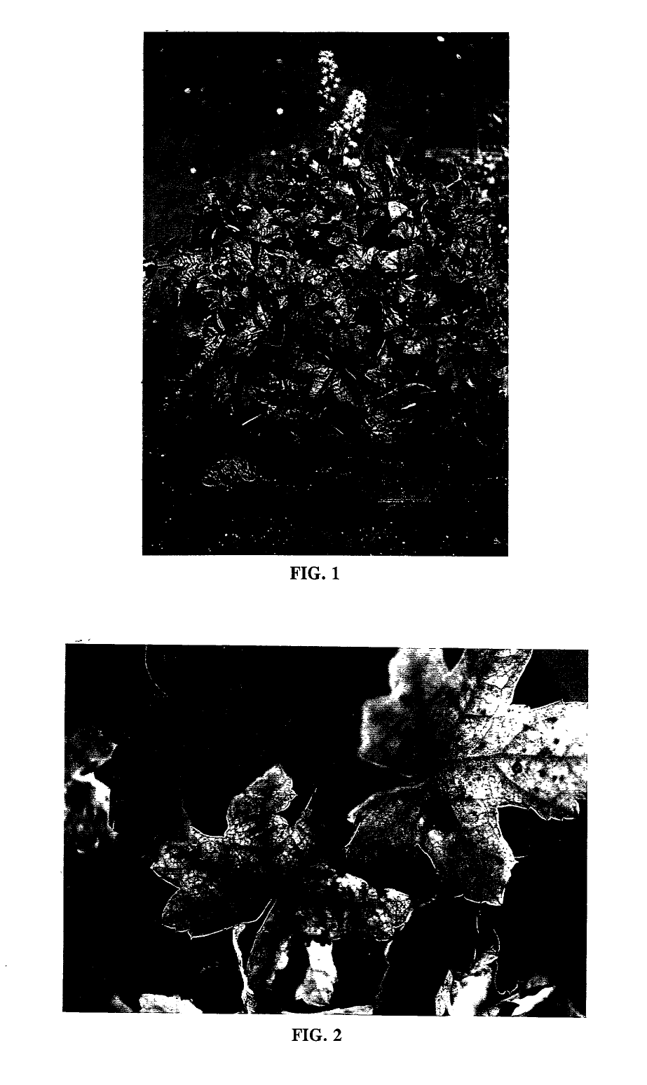

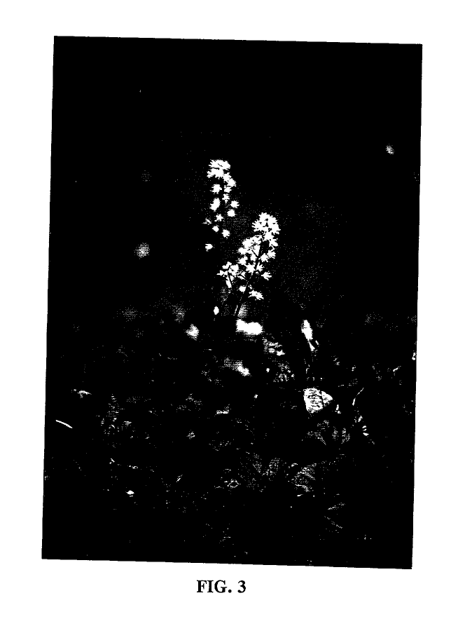

### DETAILED DESCRIPTION

The following is a detailed description of the new variety that was obtained while observing plants being grown outdoors during the summer and fall at West Grove, Pennsylvania, U.S.A. The plants were approximately two years of age and were being grown on their own roots. The chart used in the identification of color is the R.H.S. Colour Chart of The Royal Horticultural Society, London, England. More common color terms are to be accorded their ordinary dictionary significance.

| BOTANICAL CLASSIFICATION | |
|---|---|
| Tiarella cordifolia, var. collina, cv. ‘Springwood’. | |
| PLANT | |
| Habit | Compact mounding clump. |
| Type | Evergreen. |
| Height | Approximately 15 to 20 cm without blooms. |
| Width | Approximately 30 cm. |
| LEAFLETS | |
| Shape | Ovate, commonly with 5 to 7 lobes and a |
| | central lobe elongation. |
| Length | Approximately 5.5 to 9.5 cm. |
| Width | Approximately 5 to 7 cm. |
| Margins | Incised with dentation. |
| Apex | Mucronate. |
| Base | Auriculate and slightly imbricate. |
| Texture | Slightly rugose with a velvet matte finish. |
| Arrangement | Basal clump. |
| Venation | Palmately reticulate. |
| Color | Young Foliage: On the dorsal surface |
| | Yellow-Green Group 144A to 144B, and |
| | Greyed-Purple Group 187A at the center and |
| | along the main vein, and on the ventral |
| | surface Yellow-Green Group 146B to 146C. |
| | Adult Foliage: On the dorsal surface Green |
| | Group 137B to 137D, and Brown Group |
| | 200B at the center and along the main vein, |
| | and on the ventral surface Yellow-Green |
| | Group 146B to Greyed-Green Group 191A. |
| | Fall Foliage: Generally red of variable |
| | intensity as illustrated with Red Group 49D, |
| | Red Group 53D, Red-Purple Group 62D, |
| | Greyed-Purple Group 186B and Greyed- |
| | Purple Group 187B. |
| Petiole | The length commonly varies from |
| | approximately 10 to 20 cm. |
| INFLORESCENCE | |
| Type | Raceme and perfect (bisexual). |
| Number | Approximately 25 to 50 blooms per raceme. |
| Bearing | On an unbranched stalk commonly having a |
| | height of approximately 30 cm. |
| Calyx | Five-lobed. |
| Petals | Five. |
| Petal Shape | Triangular and clawed. |
| Stamens | Ten. |
| Pistil | One. |
| Flower Size | Approximately 4 to 5 mm on average per |
| | floret. |
| Color | On the dorsal surface White Group 155B and |
| | on the ventral surface White Group 155A. |
| Fragrance | Slight and sweet. |
| Pedicel | Approximately 4 mm in length on average. |
| DEVELOPMENT | |
| Vegetation | Clump-forming. |
| Blooming | Abundantly when initially blooms during |
| | May/June and sporadically thereafter during |
| | the summer and fall. |
| Resistance to | No particular susceptibility to diseases has |
| Diseases | been noted during observations to date. |

I claim:

1. A new and distinct variety of Tiarella cordifolia var. collina plant having the following combination of characteristics:
(a) exhibits a compact mounding clump growth habit with the substantial absence of runners,
(b) forms attractive white flowers on unbranched flower stalks,
(c) forms lobed ovate green leaves having a matte finish during the summer that bear somewhat maroon centers and turn red of variable intensity during the fall, and
(d) is particularly well suited for growing as a distinctive ornamental ground cover;
substantially as illustrated and described.
